(19) United States
(12) Patent Application Publication
(10) Pub. No.: US 2020/0408779 A1
(43) Pub. Date: Dec. 31, 2020

(54) DOC2B AS A BIOMARKER FOR TYPE 1 DIABETES

(71) Applicant: CITY OF HOPE, Duarte, CA (US)

(72) Inventor: Debbie C. THURMOND, Glendora, CA (US)

(73) Assignee: CITY OF HOPE, Duarte, CA (US)

(21) Appl. No.: 16/968,511

(22) PCT Filed: Feb. 8, 2019
(86) PCT No.: PCT/US2019/017364

§ 371 Date: Aug. 7, 2020

Related U.S. Application Data
(60) Provisional application No. 62/628,578, filed on Feb. 9, 2018.

(51) Int. Cl. G01N 33/68; A61K 35/39

(52) U.S. Cl. CPC G01N 33/6872; G01N 2800/042; G01N 2333/4703; A61K 35/39

(57) ABSTRACT

Disclosed is the use of DOC2B as an early stage biomarker for diagnosing type 1 diabetes (T1D) or pre-T1D or for assessing the risk of T1D or pre-T1D. Also disclosed are methods of in vivo diagnosing T1D or pre-T1D or assessing the risk of T1D or pre-T1D by detecting a reduced level of DOC2B expression in a biological sample including blood, plasm, serum, platelets, and pancreatic islets.

### PRIORITY CLAIM

This application claims priority to U.S. Provisional Application No. 62/628,578, filed Feb. 9, 2018, which is incorporated by reference herein in its entirety, including drawings.

### GOVERNMENT INTEREST

This invention was made partially with government support under Grant Nos. DK067912 and DK102233, awarded by National Institutes of Health (NIH), and under Grant Nos. 2-SRA-2015-138-S-B and 1-SRA-2016-242-Q-R, awarded by Juvenile Diabetes Research Foundation (JDRF). The Government has certain rights in the invention.

### FIELD OF THE INVENTION

The present invention relates to early detection, prevention or delaying the onset, and treatment of type 1 diabetes (T1D) or pre-T1D.

### BACKGROUND

T1D is characterized by autoimmune destruction of β-cell mass, and the preclinical phase of T1D is marked by declining β-cell function [1,2]. Studies of early interventional in T1D have shown limited effectiveness, yet have generally shown greater success in subjects that retain greater insulin secretory capacity, and in those with the shortest time since clinical onset of disease [3,4]. However, prevention efforts to protect β-cell mass are hindered by the limited availability of early biomarkers to accurately predict β-cell destruction and subsequent progression to clinical disease. Therefore, there is an unmet clinical need in detecting T1D at an early stage, preventing or delaying the onset of T1D, and treating T1D. The disclosed technology can be applied to T1D diagnosis, prognosis and treatment.

### SUMMARY OF THE INVENTION

In one aspect, disclosed herein is a method of diagnosing T1D or pre-T1D in vivo at an early stage in a subject or assessing the risk of T1D or pre-T1D in a subject. The method entails the steps of detecting the level of DOC2B expression in a biological sample collected from the subject, and comparing the level of DOC2B expression with that of a healthy, control subject or with a pre-set threshold level, wherein a reduced level of DOC2B expression indicates that the subject is suffering from or at an elevated risk of suffering from T1D or pre-T1D. In some embodiments, the biological sample includes blood, plasma, serum, platelets, and pancreatic islets. In some embodiments, detecting the level of DOC2B expression comprises detecting the level of DOC2B protein or the level of DOC2B mRNA in the biological sample. In some embodiments, the DOC2B protein level in the biological sample is determined by a high-throughput screening ELISA using one or more antibodies disclosed herein. In some embodiments, the level of DOC2B expression is reduced by at least 20%, at least 30%, at least 40%, at least 50%, at least 60%, at least 70%, at least 80%, at least 90%, or at least 95%.

In a related aspect, disclosed herein is a method of treating T1D or pre-T1D or delaying the onset of T1D or pre-T1D in a subject. The method entails the steps of detecting the level of DOC2B expression in a biological sample collected from the subject, comparing the level of DOC2B expression with that of a healthy, control subject or with a pre-set threshold level, wherein a reduced level of DOC2B expression indicates that the subject is suffering from or at an elevated risk of suffering from T1D or pre-T1D, and administering one or more T1D treatments to the subject who is determined to suffer from T1D or pre-T1D or at an elevated risk of T1D or pre-T1D. In some embodiments, the biological sample includes blood, plasma, serum, platelets, and pancreatic islets. In some embodiments, detecting the level of DOC2B expression comprises detecting the level of DOC2B protein or the level of DOC2B mRNA in the biological sample. In some embodiments, the DOC2B protein level in the biological sample is determined by a high-throughput screening ELISA using one or more antibodies disclosed herein. In some embodiments, the level of DOC2B expression is reduced by at least 20%, at least 30%, at least 40%, at least 50%, at least 60%, at least 70%, at least 80%, at least 90%, or at least 95%. In some embodiments, the one or more treatments include transplanting healthy, functional β cells or pancreatic islets to the subject.

In another aspect, disclosed herein is a method of assessing early stage pancreatic β-cell destruction or loss of functional β-cells in a subject. The method entails the steps of detecting the level of DOC2B expression in a biological sample collected from the subject, and comparing the level of DOC2B expression with that of a healthy, control subject or with a pre-set threshold level, wherein a reduced level of DOC2B expression indicates pancreatic β-cell destruction or loss of functional β-cells in the subject. In some embodiments, the biological sample includes blood, plasma, serum, platelets, and pancreatic islets. In some embodiments, detecting the level of DOC2B expression comprises detecting the level of DOC2B protein or the level of DOC2B mRNA in the biological sample. In some embodiments, the DOC2B protein level in the biological sample is determined by a high-throughput screening ELISA using one or more antibodies disclosed herein. In some embodiments, the level of DOC2B expression is reduced by at least 20%, at least 30%, at least 40%, at least 50%, at least 60%, at least 70%, at least 80%, at least 90%, or at least 95%.

In yet another related aspect, disclosed herein is an ELISA kit for detecting the DOC2B level in a biological sample obtained from a subject. The ELISA kit includes one or more antibodies disclosed herein. In some embodiments, the ELISA kit further includes reagents and/or secondary antibodies for performing the ELISA. In some embodiments, the ELISA kit further includes instructions for using the kit. In some embodiments, the biological sample includes blood, plasma, serum, platelets, and pancreatic islets. In some embodiments, the subject is at an elevated risk of T1D or pre-T1D or suffers from T1D or pre-T1D.

### BRIEF DESCRIPTION OF THE DRAWINGS

FIGS. 1A-1B show that DOC2B protein abundance was reduced in platelets of pre-diabetic NOD mice. Platelets were isolated from 16-week (FIG. 1A) or 13-week (FIG. 1B) old group-housed female NOD and age-matched NOR mice and proteins were resolved on SDS-PAGE for immunoblotting. DOC2B levels were quantified relative to tubulin immunoblotting in the same lane. Dashed vertical lines indicate splicing of lanes from within the same gel exposure. Data are shown as means±SEM (n=3-6 mice per group); *p<0.05.

FIGS. 2A-2C show that islets from young pre-diabetic NOD mice were deficient in DOC2B protein. Islets were isolated from 16-week (FIG. 2A), 13-week (FIG. 2B) or 7-week (FIG. 2C) old group-housed female NOD and age-matched NOR mice and proteins were resolved on SDS-PAGE for immunoblotting. DOC2B levels were quantified relative to tubulin loading in the same lane. Dashed vertical lines indicate splicing of lanes from within the same gel exposure. Data are shown as means±SEM for DOC2B (n=3-7 mice per group); *p<0.05.

FIG. 3 shows that DOC2B protein abundance was reduced in platelets from new-onset pediatric T1D human subjects. Platelets were isolated from new-onset T1D patients at the time of diagnosis (“Diagnosis”) and 7-10 weeks later (“First Follow-up”), and from matched controls (“Control”). Platelet proteins were resolved on SDS-PAGE for immunoblotting. Standard curves were generated using recombinantly-expressed and purified human DOC2B protein on each gel to confirm that the band intensities of DOC2B in human platelets fell within the dynamic range of the curve on the same gel. DOC2B was quantified relative to protein loading determined by Ponceau S staining in the same lane (37-68 kDa segment). Dashed vertical lines indicate splicing of lanes from within the same gel exposure. Data are shown as means±SEM for DOC2B (n=11-14 per group (gender-combined group, 8 males per group, 3-6 females per group); *p<0.05, Diagnosis vs. Control.; #p<0.05 Follow-up vs. Control).

FIGS. 4A-4B show that DOC2B protein and mRNA abundance was reduced in adult human islets subjected to treatment with pro-inflammatory cytokines. Human adult cadaveric islets were incubated under control conditions or with pro-inflammatory cytokines for 72 h at 37° C. Islet protein lysates were resolved by SDS-PAGE for immunoblotting (FIG. 4A) or for RNA extraction and qRT-PCR analysis (FIG. 4B). In addition to hDOC2B and tubulin, iNOS levels were also evaluated by immunoblotting. Bars represent mean±SEM for 4 or 5 independent sets of human islets evaluated for protein and mRNA analyses, respectively; ****p<0.0001, **p<0.002.

FIGS. 5A-5C show that DOC2B protein levels were reduced in islets from pediatric T1D humans. Slides obtained from nPOD comprised of early-onset T1D and age-matched non-diabetic human pancreata were immunostained for the presence of DOC2B, insulin or glucagon positive cells. FIG. 5A shows representative images, low power images scale bar=100 μm, higher magnification images scale bar=25 μm. FIG. 5B shows tabulated relative intensities; n=3 donors, *p<0.05. FIG. 5C shows the number of DOC2B-positive p-cells p=not significant, (N.S.).

FIGS. 6A-6B show that DOC2B levels in adult T1D human platelets were increased after clinical islet transplantation. Platelets obtained from two clinical islet transplant recipients prior to (Day 0) islet infusion, or on Day 30 and Day 75 post-infusion, were evaluated by quantitative immunoblotting for DOC2B protein content: subject COH-027 (FIG. 6A), and subject COH-028 (FIG. 6B). Ponceau S staining and GAPDH show the relative protein loading of the membranes used for immunoblotting.

FIG. 7 shows that platelet proteins from children with T1D and age/gender/BMI matched controls were isolated at Diagnosis and First Follow-up 7-10 weeks later, then resolved on SDS-PAGE for immunoblotting for STX4. Standard curves were included using recombinantly-expressed and purified human STX protein on each gel with band intensities of STX4 in human platelets falling within the dynamic range of the curve on the same gel. Dashed vertical lines indicate splicing of lanes from within the same gel exposure. Data are shown as means±SEM. n=10-13 per gender-combined group, 5-7 males per group, 3-6 females per group); *p<0.05, Diagnosis vs. Control; #p<0.05 Follow-up vs. Control.

FIG. 8 shows a diagram of the epitopes on human DOC2B.

FIG. 9 shows the alignment of DOC2B and DOC2A amino acid sequences.

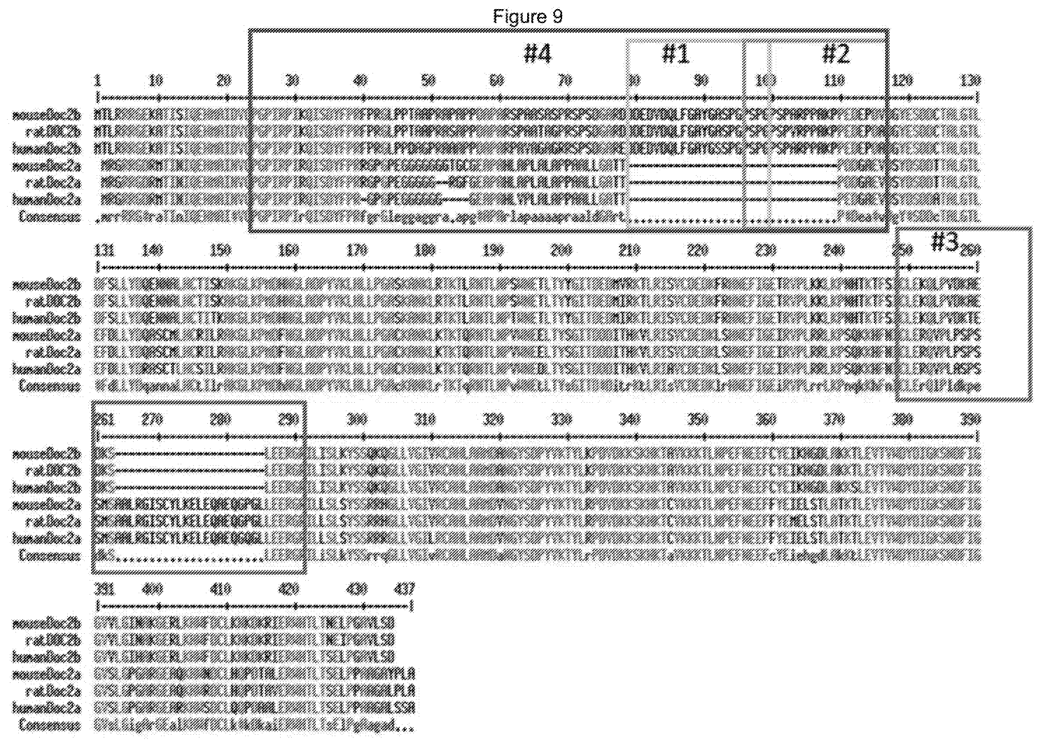

FIG. 10 shows the alignment of DOC2B amino acid sequences across species.

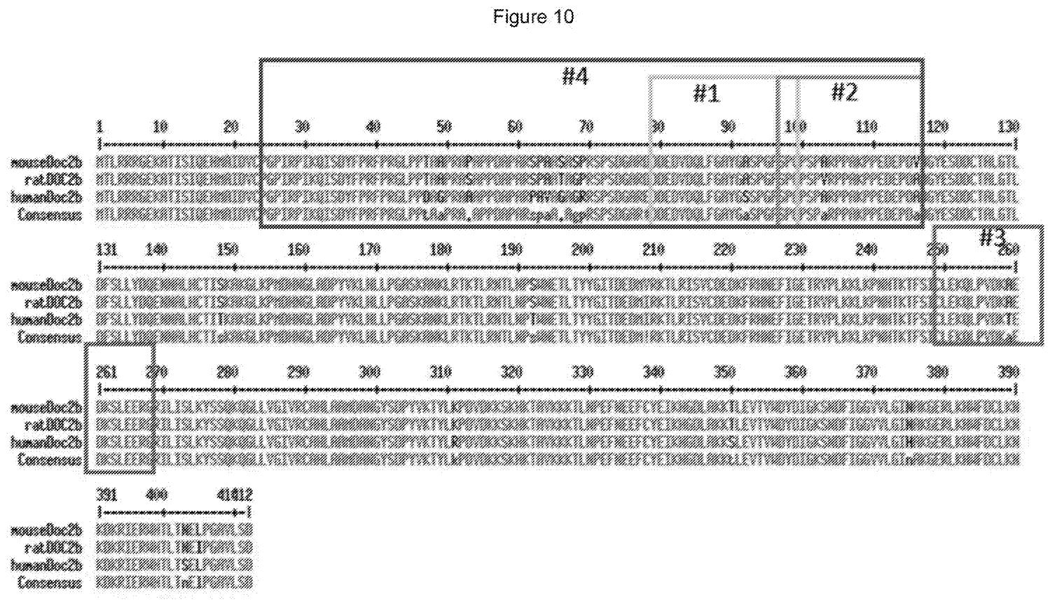

FIG. 11 shows immunofluorescent detection of DOC2B in mouse 13 cells and mouse pancreas.

FIG. 12 shows immunoblot detection of DOC2B with Antibody #2.

FIG. 13 shows immunoblot detection of DOC2B with Antibody #2 rabbit 12727 and rabbit 12728.

### DETAILED DESCRIPTION

The following description of the invention is merely intended to illustrate various embodiments of the invention. As such, the specific modifications discussed are not to be construed as limitations on the scope of the invention. It will be apparent to one skilled in the art that various equivalents, changes, and modifications may be made without departing from the scope of the invention, and it is understood that such equivalent embodiments are to be included herein.

The term “subject” or “patient” as used herein can be any individual mammal, including but not limited to human, canine, rodent, primate, swine, equine, sheep, and feline. In a particular embodiment, the subject is human.

The terms “treat,” “treating,” and “treatment” as used herein with regard to a condition refer to preventing the onset of the condition, alleviating the condition partially or entirely, or eliminating, reducing, or slowing the development of one or more symptoms associated with the condition.

Disclosed herein is a correlation between functional β-cell mass and the level of DOC2B expression in a biological sample, where the reduction of DOC2B expression indicates the loss of functional β-cell mass, thereby leading to the early diagnosis of T1D or pre-T1D. The DOC2B expression level is reduced even prior to the onset of T1D or pre-T1D. Therefore, DOC2B can be used as an early biomarker not only to report the status of T1D or pre-T1D but also to prevent or delay the onset of T1D or pre-T1D. Additionally, the DOC2B expression level in blood, plasma, serum and/or platelets closely correlates with the DOC2B expression in pancreatic islets. Therefore, the method disclosed herein allows a non-invasive, early diagnosis of T1D or pre-T1D or early assessment of T1D or pre-T1D risk from a blood, plasma, serum or platelet sample.

In healthy β-cells, insulin secretion requires soluble N-ethylmaleimide-sensitive factor-attachment protein receptor (SNARE) proteins and associated accessory regulatory proteins to promote the docking, priming, and fusion of insulin vesicles at the plasma membrane. Two target membrane (t)-SNARE proteins, Syntaxin1/4 and SNAP25/23, and one vesicle associated (v-SNARE) protein, VAMP2, constitute the SNARE core complex [5]. Assembly of the SNARE complex occurs when one v-SNARE binds two cognate t-SNARE proteins in a heterotrimeric ratio [6]. SNARE complex assembly is also facilitated by Double C2-domain protein 13 (DOC2B) [7,8]. It has been established that in animal models, deficiencies in DOC2B result in glucose intolerance and insulin secretion defects [9,10]. Conversely, overexpression of DOC2B using global transgenic mouse models enhances insulin secretion and peripheral glucose uptake [11]. Although DOC2B deficiency in rodents has been linked to T2D [12], the association between DOC2B protein levels and T1D is still unknown.

Deficient first-phase insulin secretion is a hallmark of preclinical T1D [1,2], thus, the ability to assess early pancreatic β-cell destruction is critically important for predicting disease onset. Currently, risk prediction for T1D relies heavily on family history, genetic screening, and the presence of antibodies against β-cell antigens that often appear relatively late in the progression of disease. The use of autoantibodies in evaluating T1D risk is limited, as >50% of autoantibody-positive patients remain disease-free, even at 5 years follow up [13]. Risk scores have been established [14], but remain insufficient to provide an accurate prognosis, nor an accurate measurement of β-cell health, as many autoantibody-positive individuals are slow to progress through the stages [15] of preclinical disease. To improve early prediction of T1D, ongoing studies seek to investigate the levels of circulating factors that reflect declining β-cell health, such as proinsulin [16], HSP-90 [17], and unmethylated insulin DNA [18] as potential biomarkers of T1D.

Another potential source of biomarkers is the blood-derived plasma or platelet, which is currently being investigated in diseases such as Alzheimer's disease [19] and cancer [20], and has been implicated in T1D. Changes in the platelet proteome and morphology have been noted in T1D; for instance, altered intracellular Ca²⁺ [21], enhanced formation of microparticles [22], and altered morphology [23] have been reported to result in platelet hyper-reactivity and development of vasculopathies. Importantly, platelets harbor many of the same exocytosis proteins as the pancreatic β-cell, including SNARE isoforms and regulatory accessory proteins [24].

The ability to detect β-cell destruction is critical in accurately predicting prognosis during the preclinical phase of T1D, hence the current need for additional early biomarkers. As described herein, DOC2B protein levels are substantially reduced in plasma, platelets and islets from pre-diabetic NOD mice vs. NOR control mice. Furthermore, it is shown that levels of human DOC2B are significantly lower at the time of diagnosis in plasma or platelets of new-onset T1D pediatric patients than platelets from matched control subjects. Notably, DOC2B levels are reduced at 7-10 weeks post-diagnosis, despite therapeutic remediation of hyperglycemia in the human subjects. Consistent with this, islet DOC2B protein levels are reduced in pancreatic tissue samples from T1D patients compared to matched controls. Loss of DOC2B protein and mRNA can be recapitulated by exposure of non-diabetic human islets to pro-inflammatory cytokines ex vivo, suggesting that the inflammatory milieu in pre-diabetic and T1 D humans may cause DOC2B loss. Remarkably, clinical islet transplant recipients exhibit a restoration of DOC2B levels in platelets, compared with their own nearly undetectable levels of platelet DOC2B prior to receiving the transplanted islets. These data suggest that DOC2B protein can be a biomarker of pre-diabetes and T1 D, with the levels possibly reporting relative functional p-cell mass.

Thus, biomarkers of p-cell destruction in blood have more clinical potential than those in pancreatic islets, as islet procurement is not feasible for routine diagnosis; therefore, the correlation between DOC2B protein abundance in blood-derived platelets and pancreatic islets of T1D mice and humans is investigated. As shown in the working examples, protein abundance of DOC2B is reduced in plasma, platelets and islets from humans with new-onset T1 D, compared to matched controls. DOC2B levels are substantially increased in T1 D human platelets after transplantation, when C-peptide levels are markedly increased.

As disclosed herein, an association between T1D or pre-T1D and levels of an exocytosis protein in blood-derived plasma, platelets and pancreatic islets is established. Reduced DOC2B in islets is indicative of deficient islet functional health [9]. Strikingly, plasma or platelet DOC2B levels in islet transplant recipients correlated with the presence of a functional islet mass. This correlative finding supports the possibility that the plasma or platelet DOC2B stems not necessarily from the pancreas per se, since islets are grafted into the liver in these human recipients, but that the plasma or platelets and/or precursor megakaryocytes may be sampling DOC2B from the islets irrespective of islet location. It also remains possible that the increased DOC2B content stems from “rested” native residual islets of the transplanted patients. However, this is inconsistent with the pediatric platelet data showing that even after insulin therapy to ameliorate new-onset hyperglycemia, DOC2B levels remained deficient. Mechanistically, questions arise as to how plasma, platelets and islets “communicate” to determine DOC2B levels. Supporting the concept of platelet-islet communication, it has been demonstrated that islet transplantation in T1D patients stabilizes platelet abnormalities, as transplant recipient platelets show normal volume and activation [33]. Indeed, β-cells release exosomes as a way of shuttling various miRNAs, mRNAs, and proteins to targeted peripheral cells [34]. 13-cell exosomes were also recently shown to carry proteins such as GAD-65, IA-2, and proinsulin, to dendritic cells, which then become activated [35]. Furthermore, platelets can selectively absorb proteins from the blood [36]. In fact, platelet sequestration of tumor-specific proteins was detected in animals harboring small tumors [36]. Notably, a direct interaction between platelets and pancreatic p-cells has been reported, and protein from platelets was shown to be transferred to p-cells [37].

The concept of DOC2B as a biomarker is novel because DOC2B levels in plasma, platelets and islets are significantly decreased in normoglycemic NOD mice months before their conversion to T1D. Female NOD mice typically convert to T1D between 18-24 weeks of age, but as early as 5 weeks of age, NOD mouse islets show signs of insulitis, resulting from an initial phase of pancreatic inflammation that reduces p-cell function and mass [38]. Given that DOC2B content in human islets decreased upon islet exposure to pro-inflammatory cytokines, which was sufficient to evoke iNOS expression, it is possible that the cytokine-induced drop in islet DOC2B signals reduced islet viability. Although it has been demonstrated by multiple groups that whole-body DOC2B knockout mice show deficient glucose-stimulated insulin secretion [9,10], β-cell mass was not evaluated. While it is also possible that DOC2B expression is genetically repressed in NOD mice, the genetics of NOD mice have been well studied and DOC2B was not identified as deviating from control [39]. DOC2B mRNA expression was also decreased in response to pro-inflammatory cytokine exposure in non-diabetic human islets, suggesting that DOC2B might undergo transcriptional repression during T1D development.

DOC2B protein level in a biological sample can be detected by a high-throughput screening ELISA using the antibodies disclosed herein. The ELISA has an improved accuracy and reliability due to the use of antibodies having less cross-reactivity and fewer non-specific bindings such that the assay has little or no background noise for the detection of DOC2B protein level in the sample. The ELISA results are validated by quantitative immunoblotting of known plasma samples.

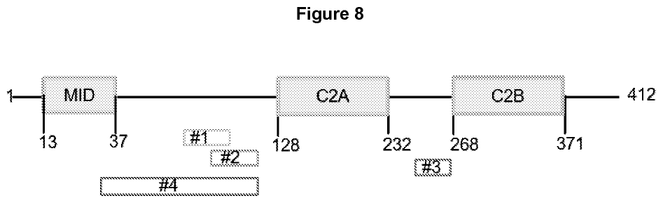

FIG. 8 illustrates the design of the antibodies used in the ELISA. Computer programs for modeling the tertiary structure of DOC2B, including alignment of C2AB containing proteins by Cluster W: information from Vaidehi's core (Supriyo) was used. The 4 antibodies disclosed herein bind to the following antigens: Antibody #1 binds to human DOC2B amino acid sequence AA 79-99, Antibody #2 binds to human DOC2B amino acid sequence AA 96-116, Antibody #3 binds to human DOC2B amino acid sequence AA 249-267 for detection of C2AB, and Antibody #4 binds to human DOC2B amino acid sequence AA 23-62, 55-92, and 82-116.

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### EXAMPLE 1

### Materials and Methods

Animals: Animals were maintained under protocols approved by the Indiana University Institutional Animal Care and Use Committee and following the National Research Council Guidelines for the Care and Use of Laboratory Animals. Female non-obese diabetic (NOD) NOD/ShiLtJ (RRID:IMSR JAX:001976) and major histocompatibility complex (MHC)-matched control non-obese diabetes resistant (NOR) (RRID:IMSR JAX:002050) mice were obtained from the Jackson Laboratory (Bar Harbor, Me.). Female NOD mice began to convert to T1D at 17-18 weeks of age, with an average conversion rate of 78% by 20 weeks of age, as previously reported [25]. Random blood glucose analysis was performed weekly to monitor conversion to T1D, which is characterized by non-fasting blood glucose levels >250 mg/dl for three consecutive days. To assess DOC2B levels before conversion to T1D, pancreatic islets were isolated, using a method as described previously [26] at 7 weeks (earliest time point for sufficient islet cell yield), 13 weeks (intermediate time point), and 16 weeks of age (latest time point before conversion to T1D). Islet isolation yield decreased in mice less than 8 weeks of age [27]. Islet lysates were then used for SDS-PAGE and immunoblotting. Mouse blood was collected and platelets were isolated as previously described [24]. Platelet lysates were then used for SDS-PAGE and immunoblotting.

Human Subjects: All human studies were conducted in keeping with the principles set out in the Declaration of Helsinki. This protocol was approved by the Indiana University Institutional Review Board. For evaluation of DOC2B levels in human platelets (new-onset T1D study), subjects aged 8-14 (11 males and 6 females) with new-onset T1D were recruited over an 18-month period. Consent was obtained from parents, with assent from the pediatric subjects. Subjects were diagnosed with T1D if they met the criteria of 1 or more positive autoantibodies with clinical features of T1D: hyperglycemia, weight loss, and normal body mass index (BMI) or those who were autoantibody negative but <10 years old at diagnosis. Exclusion criteria were as previously described [17]. For each visit, subjects received $25. Subjects had blood drawn at diagnosis and at the first follow-up appointment 7-10 weeks after diagnosis. Insulin treatment of T1D subjects was started at time of diagnosis. Non-diabetic control subjects (8 males and 6 females) were recruited from the community and matched to T1D subjects based on gender, age, and BMI (see Table 1 for demographic data).

TABLE 1
Pediatric T1D study demographics

| Characteristic | Non-T1D controls | T1D subjects |
|---|---|---|
| Number of subjects | 14 | 17 |
| Age in years, (range) | 11.4 (8.0-14.3) | 10.3 (4.3-14.1) |
| Gender (male) | 55% | 57% |
| BMI (kg/m²)* | 20.2 ± 3.0 | 17.5 ± 2.8 |
| Number of autoantibodies | — | 0 AutoAb positive: 1 |
| positive ^{†} | | 1 AutoAb positive: 5 |
| | | 2 AutoAb positive: 9 |
| | | 3 AutoAb positive: 2 |
| Basal insulin requirement prior to | — | 0.30 ± 0.09 |
| hospital discharge (units/kg/d) | | |
| C-peptide at diagnosis (pmol/l) ^{‡} | — | 110 ± 169 (13-608) |
| HbA1c at diagnosis (range) | — | 11.0 ± 1.7% |
| | | (7.5 ± 14.2) |
| HbA1c at first follow-up (range) | — | 7.7 ± 0.8% (6.4-9.0) |

Abbreviations:
BMI, body mass index;
HbA1c, hemoglobin A1c;
T1D, type one diabetes.
Values displayed are means ± SD unless otherwise noted.
*For BMI calculations, 1 T1D subject did not have a diagnosis height and 1 non-T1D control did not have a registration height. For these subjects, the heights from clinic follow-up were used to calculate BMI.
^{†} The following 3 diabetes-associated antibodies were tested: GAD, miAA, and IA-2A.
^{‡} For C-peptide at diagnosis, n = 13.

Samples were de-identified and coded by the clinical team prior to distribution to the research lab for platelet isolation and analyses. Platelets were isolated by centrifugation from blood, as previously described [28], and lysed for SDS-PAGE and immunoblotting. Upon quantification of the data for each sample, the clinical team re-identified samples to permit grouping of data into T1D vs. non-diabetic for statistical comparisons.

For evaluation of DOC2B levels in human islets (T1D islet transplantation study), samples were obtained from T1D islet transplantation recipients, as approved by the City of Hope Institutional Review Board. Two subjects, aged 43 and 52 years, were recruited for human islet transplantation based on the following criteria: T1D diagnosis with frequent or life-threatening hypoglycemia with or without unawareness symptoms. Blood was obtained from both subjects prior to transplantation (Day 0), and on Day 30 and Day 75 after islet transplantation (see Table 2 for demographic data).

TABLE 2
Baseline adult islet transplant recipient and islet graft characteristics

| Characteristics | COH-027 | COH-028 |
|---|---|---|
| Recipient Characteristics | | |
| Gender | F | M |
| Age at transplant (years) | 43 | 52 |
| Weight (kg) | 76.5 | 92 |
| BMI (kg/m²) | 28.93 | 29.77 |
| Duration of diabetes | 33 | 34 |
| (years) | | |
| HbA1c (%) | 5.5 | 8.5 |
| Insulin intake (units/day) | 28 | 52 |
| Fasting/glucagon- | 0.03/0.02 | <0.02/<0.02 |
| stimulated C-peptide | | |
| (ng/ml) | | |
| Autoantibodies | GAD65-neg \| IA-2-pos | GAD65-pos \| IA-2-neg |
| | mIAA-pos \| ZnT8-neg | mIAA-pos \| ZnT8-neg |
| PRA class I/class II (%) | 0/0 | 0/0 |
| Islet Graft Characteristics | | |
| Total islet dose (IEQ) | 240,133 | 482,755 |
| IEQ/kg | 3,139 | 5,247 |
| Islet purity (%) | 50 | 68 |
| Packed cell volume (ml) | 1.9 | 2.8 |
| Islet viability (%) | 91 | 94 |

Abbreviations:
PRA, panel reactive antibody;
IEQ, islet equivalent.

Platelets were isolated by centrifugation from blood, as previously described [28], and lysed for SDS-PAGE and immunoblotting.

Islet cell transplantation: For the T1D islet transplant study, human pancreata were procured from ABO-compatible, cross-match negative cadaveric donors. The islets were isolated under cGMP conditions by the Southern California Islet Cell Resource Center at City of Hope using a modified Ricordi method. Islets were maintained in culture for up to 72 hours prior to transplantation. Islets were transplanted intraportally with heparinized saline (35 U/kg recipient body weight) using a transhepatic percutaneous approach.

Clinical/laboratory assays: For the new-onset T1D study, autoantibodies to glutamic acid decarboxylase 65 (GAD-65), insulin, and Islet Antigen 2 (IA2) were assayed from peripheral blood at diagnosis at Mayo Medical Laboratories (Rochester, Minn.). Glycated hemoglobin (HbA1c) was also measured at diagnosis and at first clinic follow-up (7-10 weeks after diagnosis) using the Bayer A1cNow system or the Bayer DCA2000 analyzer (Tarrytown, N.Y.). C-peptide was measured in stored serum samples using the C-peptide ELISA kit (Alpco, Salem, N.H.; detection range 20-3000 pM).

For the T1D islet transplant study, plasma C-peptide measurements were performed by the Northwest Lipid Metabolism and Diabetes Laboratory (Seattle, Wash.) using the Tosoh C-Peptide II Assay (Tosoh Bioscience, Inc, San Francisco, Calif.; detection range 0.02-30 ng/ml). A fasting C-peptide <0.2 ng/ml and 6-min glucagon-stimulated C-peptide <0.3 ng/ml were used to confirm T1D diagnosis prior to islet transplant. Autoantibodies (GAD-65, IA-2A, insulin [m IAA], and zinc transporter 8 [ZnT8]) were analyzed using radiobinding assays by the Autoantibody/HLA Service Center at the Barbara Davis Center for Diabetes (Aurora, Calif.).

Ex vivo islet preparations: Non-T1D human cadaveric pancreatic islets were obtained through the Integrated Islet Distribution Program at City of Hope. The islets were prepared and treated with a cytokine mixture (10 ng/ml TNF-α, 100 ng/ml IFN-γ and 5 ng/ml IL-1β; ProSpec, East Brunswick, N.J., USA) for 72 hours, as previously described [29]. The islets were then used in qRT-PCR analysis or SDS-PAGE followed by immunoblotting.

Immunofluorescence: Human paraffin-embedded pancreatic tissue sections were obtained from the Network for Pancreatic Organ Donors with Diabetes (nPOD). Five sections from formalin-fixed paraffin-embedded (FFPE) tissue samples were obtained from T1D (n=3) and age and BMI-matched non-diabetic (n=3) donors. Pancreas sections were immunostained with primary and secondary antibodies listed in Table 3.

TABLE 3
Primary and secondary antibodies used in study

| | | | |
|---|---|---|---|
| Protein Target | Source | Catalogue No. | RRID No. |

Primary antibodies used in NOD mouse study/ex vivo stet study

| | | | |
|---|---|---|---|
| DOC2B | Proteintech | 20574-1-AP | AB_10696316 |
| Tubulin | Abcam | ab56676 | AB_945996 |
| iNOS | Millipore | ABN26 | AB_10805939 |

Primary antibodies used in New-onset T1D/T1D transplant study

| | | | |
|---|---|---|---|
| DOC2B | Abnova | H00008447-B01P | AB_10549446 |
| GAPDH | Abnova | ab9485 | AB_307275 |

Primary antibodies used in immunofluorescence study

| | | | |
|---|---|---|---|
| DOC2B | Proteintech | 20574-1-AP | AB_10696316 |
| Insulin | Abcam | ab7842) | AB_306130 |

Secondary antibodies

| | | | |
|---|---|---|---|
| goat anti-rabbit | Bio-Rad | 1706515 | AB_11125142 |
| goat anti-mouse | Bio-Rad | 1706516 | AB_11125547 |
| Alexa Fluor 568 | Abcam | ab175471 | AB_2576207 |
| goat anti-rabbit | | | |
| Alexa Fluor 488 | Thermo | A-11073 | AB_2534117 |
| goat anti-guinea pig | | | |

Slides were counterstained to mark the nuclei, using 4′,6-diamidino-2-phenylindole (DAPI) (Vectashield; Vector Laboratories, Burlingame, Calif.) and viewed using a Keyence BZ X-700 fluorescence microscope (Keyence Corporation, Itasca, Ill.). All human T1D samples were prepared and processed at the same time; confocal images were taken with identical acquisition settings. Islet immunofluorescence was assessed by imaging 20-30 islets (grouping of four or more insulin-positive cells) per subject. Analysis was performed in a blinded fashion using Image-Pro Software (Media Cybernetics, Rockville, Md., USA) to quantify fluorescence intensities using methods as previously described [30]. Defined regions of interest (ROIs) were used to delimit islets from adjacent acinar tissue and average intensity measurements of insulin and DOC2B were quantified by splitting the merged image into two color channels with the same ROI.

Immunoblotting: Platelet and islet protein lysates for the NOD mouse study were resolved on a 10% SDS-PAGE gel and transferred to standard PVDF (Bio-Rad, Hercules, Calif., USA). Platelet proteins from the new-onset T1D study were resolved on a 10% SDS-PAGE gel using an SE400 air-cooled 18×16 cm vertical protein electrophoresis unit (Hoefer, Inc. Holliston, Mass.) and transferred to standard PVDF (Bio-Rad). Platelet proteins from the T1D islet transplant study were resolved on a 12% SDS-PAGE gel using a Criterion™ 13.3×8.7 cm vertical electrophoresis unit (Bio-Rad) and transferred to standard PVDF. All blots were probed as outlined in Table 3.

Quantitative real-time PCR: Total RNA was isolated from human islets using the Qiagen RNeasy Plus Mini Kit (Qiagen, Valencia, Calif., USA) and assessed using the QuantiTect SYBR Green RT-PCR kit (Qiagen). Primers used for the detection of hDoc2b are as follows: forward: 5′-CCAGTAAGGCAAATAAGCTC-3′ and reverse: 5′-GGGTTTCAGCTTCTTCA-3′. Standard tubulin primers (Cat: QT00089775, Qiagen) were used for normalization.

Statistical analysis: Data were evaluated for statistical significance using Student's t test for comparison of two groups; ANOVA and Tukey's post-hoc tests (GraphPad Software, La Jolla, Calif., USA) were used for comparison of more than two groups. Data are expressed as the average±SEM.

### EXAMPLE 2

### Low DOC2B Levels in Pre-Diabetic NOD Mouse Platelets and Islets

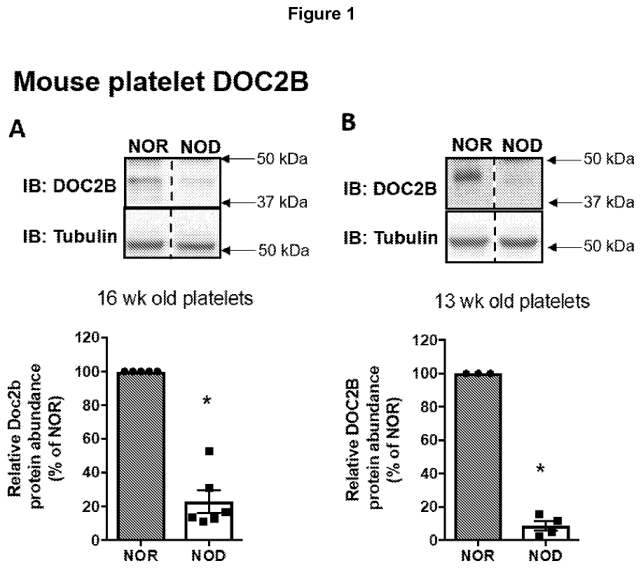

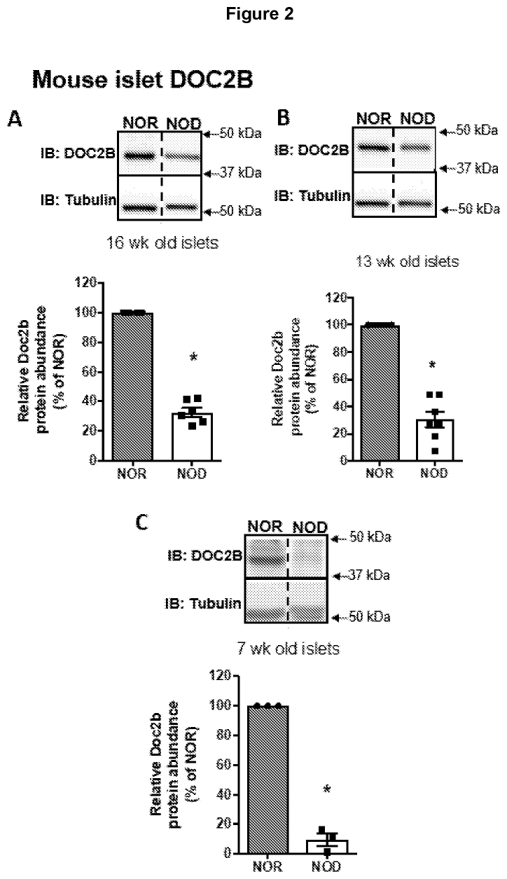

To investigate whether DOC2B protein levels are altered in the blood prior to onset of T1D, platelet DOC2B abundance in young pre-diabetic NOD mice and MHC-matched NOR mice was examined. Immunoblotting revealed that platelets from 16- and 13-week old NOD mice exhibited up to a 90% reduction in DOC2B protein levels (FIG. 1) compared to NOR platelets. Furthermore, islets from 16- and 13-week old NOD mice showed at least a 65% reduction in DOC2B protein levels (FIG. 2) compared to NOR islets. NOD islets from as early as 7 weeks of age showed a 90% reduction in DOC2B protein (FIG. 2). The average blood glucose levels from random blood testing of NOD and NOR mice were below 250 mg/dL at 7, 13, and 16 weeks (Table 4), indicating that the mice had not yet converted to diabetes. These data show that DOC2B protein abundance is reduced in both islets and platelets of prediabetic mice.

TABLE 4
Average blood glucose levels of NOD and NOR
mice at 16, 13, and 7 weeks
Avg. Non-fasting Blood Glucose (mg/dL)

| | 16 weeks | 13 weeks | 7 weeks |
|---|---|---|---|
| NOR | 197 ± 17 | 183 ± 10 | 130 ± 6 |
| NOD | 196 ± 19 | 194 ± 22 | 127 ± 5 |

Data represent the average ± S.E; n = 6 per group for mice at 16 and 13 weeks; n = 5 per group at 7 weeks. Random non-fasting blood glucose was measured for NOR and NOD female mice at 13 and 16 weeks of age. No statistical differences were seen.

### EXAMPLE 3

### Low DOC2B Levels in New-Onset T1D Human Platelets

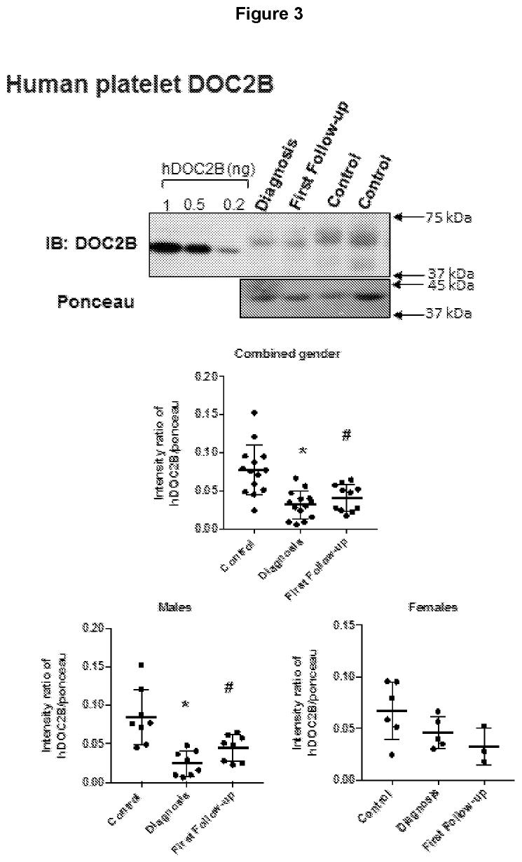

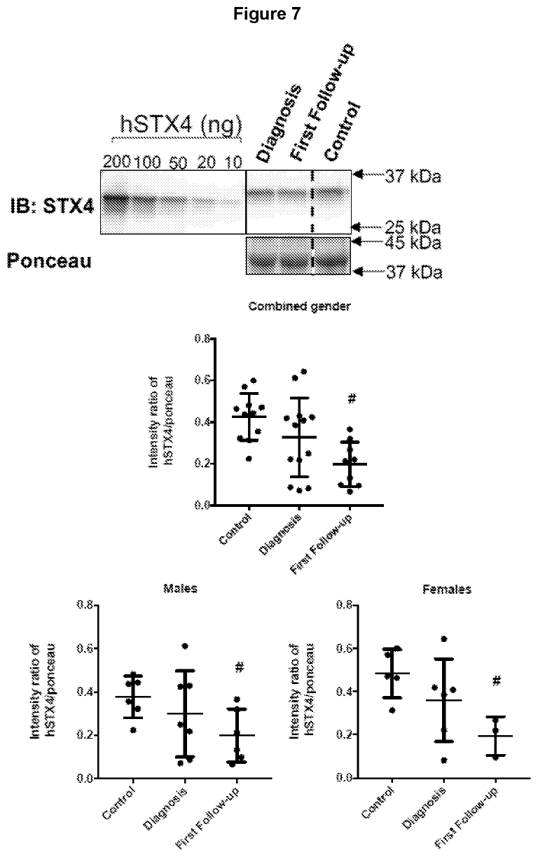

In the new-onset T1D study, the protein content of DOC2B was quantified using platelets from new-onset T1D subjects in comparison to controls (Table 1). Platelets from new-onset T1 D subjects exhibited reduced protein levels of DOC2B for both genders, both at diagnosis and at first clinic follow-up 7-10 weeks later. When males and females were assessed separately, DOC2B levels were reduced in males by ˜70% compared to non-diabetic control subjects, persisting even after insulin treatment of the patient and reduction of HbA1c (FIG. 3). The significant loss of DOC2B at T1 D diagnosis was selective for DOC2B compared to another exocytosis protein, syntaxin 4 (STX4) (FIG. 7). These data indicate that DOC2B was decreased in T1 D platelets independent of glycemic control, relative to non-diabetic human platelets, and that platelet DOC2B levels were already diminished at T1D diagnosis.

### EXAMPLE 4

### Ex Vivo Pro-Inflammatory Cytokines Treatment Reduces Human Islet DOC2B Levels

T1D is associated with elevated circulating pro-inflammatory cytokines, which damages p-cells [31]. Because obtaining pancreatic islets from living T1 D subjects is virtually impossible, the relationship between T1 D and DOC2B levels was evaluated by treating human cadaveric non-diabetic islets (Table 5) ex vivo with pro-inflammatory cytokines in effort to simulate the circulating milieu.

TABLE 5
Non-diabetic human islet donor characteristics

| Unos/ | | | | | | Islet | |
|---|---|---|---|---|---|---|---|
| COH | | | | | Islet | Viabil- | Exp. |
| ID no. | Sex | Age | BMI | Race | Purity | ity | Use |
| ACIN402 | M | 49 | 40.0 | Hispanic | N/A | N/A | protein |
| ACIY103 | M | 24 | 24.6 | Caucasian | 78% | N/A | protein |
| Hu966 | M | 20 | 30.6 | African | 88% | N/A | protein |
| | | | | American | | | |
| ADBL | F | 53 | 23.8 | Caucasian | 90% | 90% | protein |
| ADFE489 | F | 45 | 23.1 | Asian | N/A | N/A | mRNA |
| ADDV480 | M | 52 | 25.4 | Caucasian | N/A | N/A | mRNA |
| AEFU443 | F | 49 | 29 | Caucasian | 95% | 95% | mRNA |
| Hu1000 | M | 49 | 29 | Hispanic | N/A | N/A | mRNA |
| Hu966 | M | 20 | 30.6 | African | 88% | N/A | mRNA |
| | | | | American | | | |
| ACIY103 | M | 24 | 24.6 | Caucasian | 78% | N/A | mRNA |

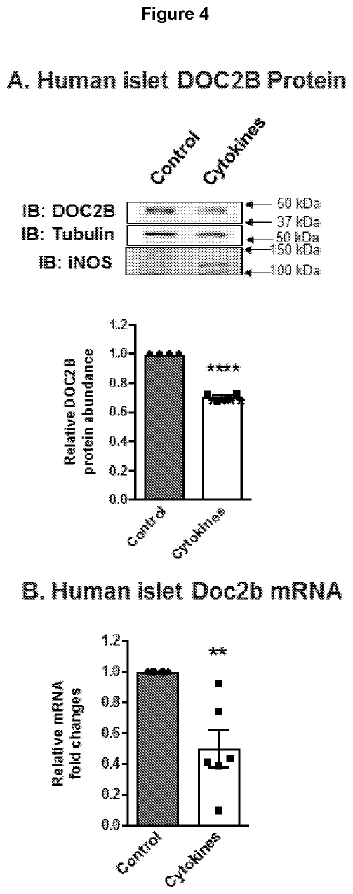

Cytokine treatment (IL-1β, TNF-α, INF-γ) elevated the levels of islet iNOS, consistent with the reported effects of cytokine exposure [32]. Correspondingly, DOC2B protein and mRNA levels were reduced by 30% and 50%, respectively (FIGS. 4A-4B). These data suggest that a T1D-like milieu can decrease DOC2B levels in human islets.

### EXAMPLE 5

### Reduced DOC2B Protein in Human Early-Onset T1 D Islets

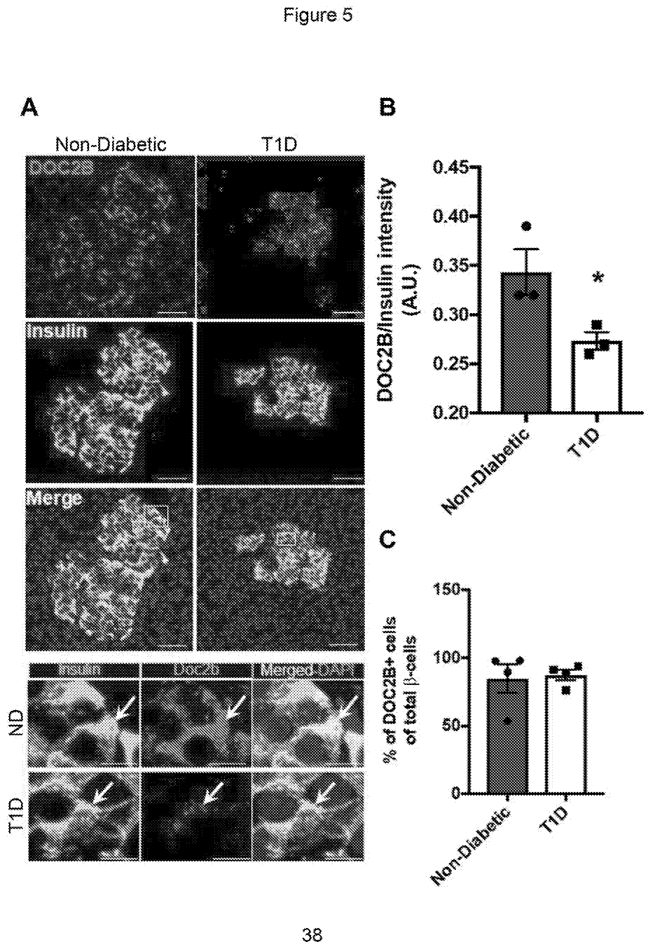

To investigate changes in DOC2B levels in T1D human pancreata, paraffin embedded slides (obtained from nPOD) from cadaveric donors were used for DOC2B immunofluorescence evaluation in early-onset pediatric T1D (5 years or less with T1D) (n=3) versus matched controls (n=3) (FIG. 5A, and Table 6).

TABLE 6
nPOD sample human pancreata donor characteristics

| nPOD ID no. | Sex | Age | BMI | Race | Years of T1D |
|---|---|---|---|---|---|
| 6113 | F | 13.1 | 24.7 | Caucasian | 1.5 |
| 6342 | F | 14 | 24.3 | Caucasian | 2 |
| 6243 | M | 13 | 21.3 | Caucasian | 5 |
| 6386 | M | 14 | 23.9 | Caucasian | ND |
| 6392 | M | 14.1 | 23.6 | Caucasian | ND |
| 6340 | M | 9.7 | 20.3 | Caucasian | ND |

Abbreviations:
ND; non-diabetic.

By measuring relative immunofluorescent intensities, a decrease in DOC2B abundance in T1D islets versus that in non-diabetic controls was detected (FIG. 5B). Although the relative number of DOC2B-positive β-cells in non-diabetic and T1D islets were similar (FIG. 5C), DOC2B intensity was reduced in T1D β-cells.

### EXAMPLE 6

### DOC2B Levels are Restored After Clinical Islet Transplantation

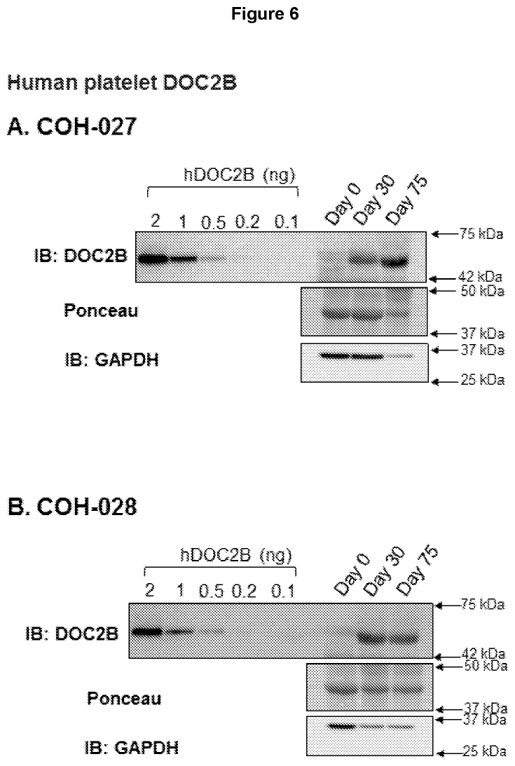

In the T1D islet transplantation study (Table 2), the pre-transplant platelet DOC2B levels were very low in both subjects relative to an hDOC2B protein standard curve (FIGS. 6A-6B, Day 0). Notably, within 30 days of transplantation, each T1D islet recipient showed a robust increase in platelet DOC2B protein, which persisted to 75 days after transplantation (FIGS. 6A-6B, Days 30 and 75). These data coincide with changes in C-peptide levels in these subjects: while each subject had low to almost undetectable fasting/glucagon-stimulated C-peptide levels before transplantation, the C-peptide levels were substantially increased by 30 days after transplantation (Table 7). As C-peptide levels are indicative of overall islet function, these data suggest that in humans, DOC2B levels in platelets correlate with relative functional p-cell mass.

TABLE 7
Islet transplant recipient treatment and outcome summary

| | | islet Transplant Recipients | |
|---|---|---|---|
| | | COH-027 | COH-028 |
| Immunosuppression Regimen | | Induction: | Induction: |
| | | rATG, etanercept, anakinra | rATG, etanercept, anakinra, |
| | | Maintenance: | Maintenance: |
| | | tacrolimus, MMF, +/−sirolimus | tacrolimus, MMF +/−sirolimus |
| Additional Immunosuppression for | | Solumedrol, Plasmapheresis, IVig & | NA |
| suspected islet graft rejection | | Rituxan for suspected islet graft | |
| | | rejection | |
| HbA1c (%) | Pre-Tx | 5.5 | 8.5 |
| | Day 30 | ND | ND |
| | Day 75 | 5.3 | 6 |
| Insulin Intake (units/day) | Pre-Tx | 28 | 52 |
| | Day 30 | 13 | 28 |
| | Day 75 | 15 | 8 |
| Fasting/Glucagon-Stimulated | Pre-Tx | 0.03/0.02 | <0.02/<0.02 |
| C-peptide (ng/ml) | Day 30 | 2.78/3.56 | 1.71/3.39 |
| | Day 75 | 0.8.4/1.42 | 12.3/2.40 |
| Mixed Meal Tolerance Test (MMTT) | Pre-Tx | ND | ND |
| C-peptide at 0/90 min | Day 30 | ND | ND |
| | Day 75 | 0.63/2.65 | 1.62/2.95 |
| Oral Glucose Tolerance Test (OGTT) | Pre-Tx | ND | ND |
| BG (mg/dl) \| C-pep (ng/ml , | Day 30 | BG.: 102/197 \| C-pep: 1.49/6.72 | BG: 127/228 \| C-p: 1.65/4.21 |
| Fasting/120 min | Day 75 | BG: 101/199 \| C-pep: 0.71/3.52 | BG: 148/310 \| C-p: 1.46/3.22 |
| Autoantibodies | Pre-Tx | GAD65-neg/IA-2-pos | GAD65-pos/IA-2-neg |
| | | mIAA-pos/ZnT8-neg | rnIAA-pos/ZnT8-neg |
| | Day 75 | GAD65-pos*/IA-2-pos | GAD65-pos / IA-2-neg |

| | |
|---|---|
| mIAA-neg*/ZnT8-neg | mIAA-pos/ZnT8-neg |

NA = Not applicable;
ND = Not done;
Pre-TX: Pre-Transpiant (baseline);
*Denotes change in auto or allo-antibodies status from baseline

### EXAMPLE 7

### DOC2B Antibodies Test Results

Four anti-DOC2B antibodies were developed: Antibody #1 binds to human DOC2B amino acid sequence AA 79-99, Antibody #2 binds to human DOC2B amino acid sequence AA 96-116, Antibody #3 binds to human DOC2B amino acid sequence AA 249-267 for detection of C2AB, and Antibody #4 binds to human DOC2B amino acid sequence AA 23-62, 55-92, and 82-116. Table 8 below shows the immunoblotting results.

TABLE 8
Immunoblotting

| | | | | | | | | | | IF: | IF: |
|---|---|---|---|---|---|---|---|---|---|---|---|
| | | recomb | human | | C2AB | Human | | human | IF: | Mouse | Human |
| Antigen | Ab | Doc2b | EndoC bH1 | MIN6 | domain | Platelet | Doc2a | plasma | MIN6 | pancreas | pancreas |
| 79-99 | #1 | +++ | ? | ? | ND | ? | ND | ? | ND | ND | NA |
| 96-116 | #2 | ++ | +++ | +++ | ND | +++ | ND | ? | ++ | + | + |
| 249-267 | #3 | + | ++ | ++ | +++ | ++ | detects | +++ | ++ | ++ | ++ |
| 23-116 | #4 | +++ | ++ | ++ | ? | ? | detects | ? | ++ | ? (BP) | ? (BP) |

Based on 1:1000 dilution; ND: not detected; NA: not applicable; ?: multiple bands; (BP): competitive binding peptide test not yet processed.

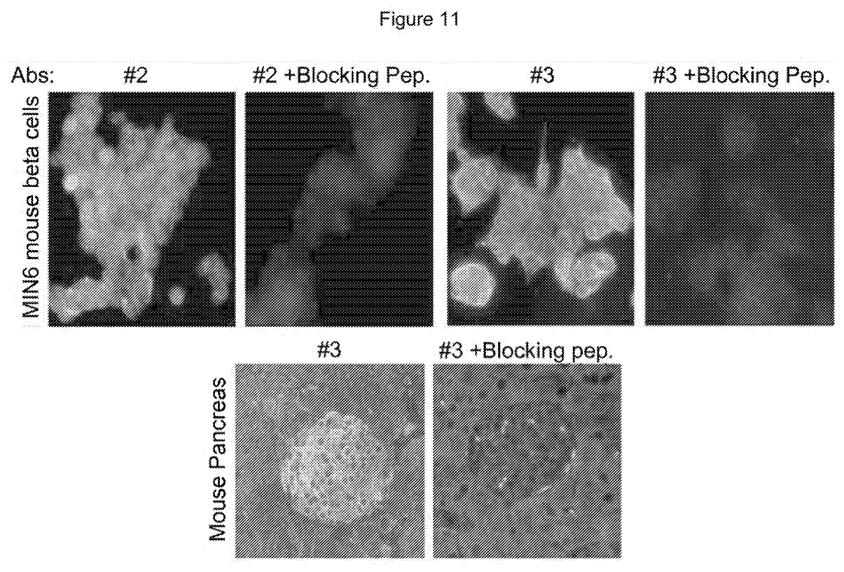

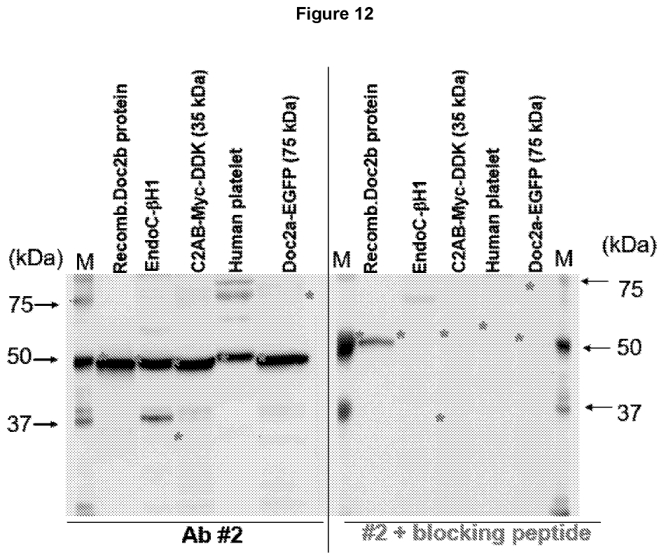

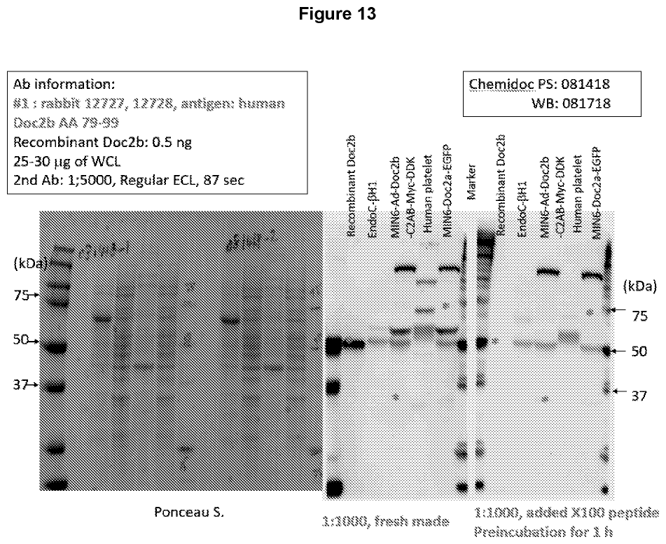

FIG. 11 shows that the antibodies disclosed herein can be detected in β cells by immunofluorescent detection. FIGS. 12 and 13 show the immunoblot detection of DOC2B Antibody #2. In FIG. 12, affinity purified Ab#2 was used at 1,000 dilution to detect endogenous Doc2b present in a variety of cell lysates. Each lane of the 10% SDS-PAGE was loaded with 25-30 mg of cell lysates indicated, proteins resolved were transferred to PVDF and used for immunoblot. Following 1 h incubation with Ab#2 at 1,000 dilution, the PVDF was washed three times with TBS-Tween for a total of 30 min at RT, then probed with a secondary antibody at a dilution of 1:5,000 for 1 h RT, and detection of bands using enhanced chemiluminescence (ECL, 45 sec exposure shown). In FIG. 13, affinity purified Ab#3 was used similarly to that of Ab#2, the only other difference being ECL detection for 87 sec.

As stated above, the foregoing is merely intended to illustrate the various embodiments of the present invention. As such, the specific modifications discussed above are not to be construed as limitations on the scope of the invention. It will be apparent to one skilled in the art that various equivalents, changes, and modifications may be made without departing from the scope of the invention, and it is understood that such equivalent embodiments are to be included herein. All references cited herein are incorporated by reference as if fully set forth herein.

### REFERENCES

1. Ferrannini E, Mari A, Nofrate V, Sosenko J M, Skyler J S. Progression to diabetes in relatives of type 1 diabetic patients: mechanisms and mode of onset. Diabetes. 2010; 59:679-685.
2. Siljander H T, Hermann R, Hekkala A, Lande J, Tanner L, Keskinen P, Ilonen J, Simell O, Veijola R, Knip M. Insulin secretion and sensitivity in the prediction of type 1 diabetes in children with advanced beta-cell autoimmunity. Eur J Endocrinol. 2013; 169:479-485.
3. Keymeulen B, Vandemeulebroucke E, Ziegler A G, Mathieu C, Kaufman L, Hale G, Gorus F, Goldman M, Walter M, Candon S, Schandene L, Crenier L, De Block C, Seigneurin J M, De Pauw P, Pierard D, Weets I, Rebello P, Bird P, Berrie E, Frewin M, Waldmann H, Bach J F, Pipeleers D, Chatenoud L. Insulin needs after CD3-antibody therapy in new-onset type 1 diabetes. N Engl J Med. 2005; 352:2598-2608.
4. Herold K C, Gitelman S E, Willi S M, Gottlieb P A, Waldron-Lynch F, Devine L, Sherr J, Rosenthal S M, Adi S, Jalaludin M Y, Michels A W, Dziura J, Bluestone J A. Teplizumab treatment may improve C-peptide responses in participants with type 1 diabetes after the new-onset period: a randomised controlled trial. Diabetologia. 2013; 56:391-400.
5. Gaisano H Y. Recent new insights into the role of SNARE and associated proteins in insulin granule exocytosis. Diabetes Obes Metab. 2017; 19 Suppl 1:115-123.
6. Kiraly-Borri C E, Morgan A, Burgoyne R D, Weller U, Wollheim C B, Lang J. Soluble N-ethylmaleimide-sensitive-factor attachment protein and N-ethylmaleimide-insensitive factors are required for Ca2+-stimulated exocytosis of insulin. Biochem J. 1996; 314:199-203.
7. Miyazaki M, Emoto M, Fukuda N, Hatanaka M, Taguchi A, Miyamoto S, Tanizawa Y. DOC2b is a SNARE regulator of glucose-stimulated delayed insulin secretion. Biochem Biophys Res Commun. 2009; 384:461-465.
8. Ke B, Oh E, Thurmond D C. Doc2beta is a novel Munc18c-interacting partner and positive effector of syntaxin 4-mediated exocytosis. J Biol Chem. 2007; 282:21786-21797.
9. Ramalingam L, Oh E, Yoder S M, Brozinick J T, Kalwat M A, Groffen A J, Verhage M, Thurmond D C. Doc2b is a key effector of insulin secretion and skeletal muscle insulin sensitivity. Diabetes. 2012; 61:2424-2432.
10. Li J, Cantley J, Burchfield J G, Meoli C C, Stockli J, Whitworth P T, Pant H, Chaudhuri R, Groffen A J, Verhage M, James D E. DOC2 isoforms play dual roles in insulin secretion and insulin-stimulated glucose uptake. Diabetologia. 2014; 57:2173-2182.
11. Ramalingam L, Oh E, Thurmond D C. Doc2b enrichment enhances glucose homeostasis in mice via potentiation of insulin secretion and peripheral insulin sensitivity. Diabetologia. 2014; 57:1476-1484.
12. Keller M P, Choi Y, Wang P, Davis D B, Rabaglia M E, Oler A T, Stapleton D S, Argmann C, Schueler K L, Edwards S, Steinberg H A, Chaibub Neto E, Kleinhanz R, Turner S, Hellerstein M K, Schadt E E, Yandell B S, Kendziorski C, Attie A D. A gene expression network model of type 2 diabetes links cell cycle regulation in islets with diabetes susceptibility. Genome Res. 2008; 18:706-716.
13. Ziegler A G, Rewers M, Simell O, Simell T, Lempainen J, Steck A, Winkler C, Ilonen J, Veijola R, Knip M, Bonifacio E, Eisenbarth G S. Seroconversion to multiple islet autoantibodies and risk of progression to diabetes in children. Jama. 2013; 309:2473-2479.
14. Sosenko J M, Krischer J P, Palmer J P, Mahon J, Cowie C, Greenbaum C J, Cuthbertson D, Lachin J M, Skyler J S. A risk score for type 1 diabetes derived from autoantibody-positive participants in the diabetes prevention trial-type 1. Diabetes Care. 2008; 31:528-533.
15. Insel R A, Dunne J L, Atkinson M A, Chiang J L, Dabelea D, Gottlieb P A, Greenbaum C J, Herold K C, Krischer J P, Lernmark A, Ratner R E, Rewers M J, Schatz D A, Skyler J S, Sosenko J M, Ziegler A G. Staging presymptomatic type 1 diabetes: a scientific statement of JDRF, the Endocrine Society, and the American Diabetes Association. Diabetes Care. 2015; 38:1964-1974.
16. Sims E K, Chaudhry Z, Watkins R, Syed F, Blum J, Ouyang F, Perkins S M, Mirmira R G, Sosenko J, DiMeglio L A, Evans-Molina C. Elevations in the Fasting Serum Proinsulin-to-C-Peptide Ratio Precede the Onset of Type 1 Diabetes. Diabetes Care. 2016; 39:1519-1526.
17. Watkins R A, Evans-Molina C, Terrell J K, Day K H, Guindon L, Restrepo I A, Mirmira R G, Blum J S, DiMeglio L A. Proinsulin and heat shock protein 90 as biomarkers of beta-cell stress in the early period after onset of type 1 diabetes. Transl Res. 2016; 168:96-106.e101.
18. Fisher M M, Watkins R A, Blum J, Evans-Molina C, Chalasani N, DiMeglio L A, Mather K J, Tersey S A, Mirmira R G. Elevations in Circulating Methylated and Unmethylated Preproinsulin DNA in New-Onset Type 1 Diabetes. Diabetes. 2015; 64:3867-3872.
19. Tang K, Hynan L S, Baskin F, Rosenberg R N. Platelet amyloid precursor protein processing: a bio-marker for Alzheimer's disease. J Neurol Sci. 2006; 240:53-58.
20. Peterson J E, Zurakowski D, Italiano J E, Jr., Michel L V, Connors S, Oenick M, D'Amato R J, Klement G L, Folkman J. VEGF, PF4 and PDGF are elevated in platelets of colorectal cancer patients. Angiogenesis. 2012; 15:265-273.
21. Zheng Y, Wang L, Zhu Z, Yan X, Zhang L, Xu P, Luo D. Altered platelet calsequestrin abundance, Na(+)/Ca(2)(+) exchange and Ca(2)(+) signaling responses with the progression of diabetes mellitus. Thromb Res. 2014; 134:674-681.
22. Witas H W, Rozalski M, Watala C, Mlynarski W, Sychowski R, Bodalski J. Do determinants of platelet function co-segregate with genetic markers of type 1 diabetes mellitus? Platelets. 1999; 10:169-177.
23. Malachowska B, Tomasik B, Szadkowska A, Baranowska-Jazwiecka A, Wegner O, Mlynarski W, Fendler W. Altered platelets' morphological parameters in children with type 1 diabetes—a case-control study. BMC Endocr Disord. 2015; 15:17.
24. Ye S, Karim Z A, Al Hawas R, Pessin J E, Filipovich A H, Whiteheart S W. Syntaxin-11, but not syntaxin-2 or syntaxin-4, is required for platelet secretion. Blood. 2012; 120:2484-2492.
25. Tersey S A, Nishiki Y, Templin A T, Cabrera S M, Stull N D, Colvin S C, Evans-Molina C, Rickus J L, Maier B, Mirmira R G. Islet beta-cell endoplasmic reticulum stress precedes the onset of type 1 diabetes in the nonobese diabetic mouse model. Diabetes. 2012; 61:818-827.
26. Oh E, Stull N D, Mirmira R G, Thurmond D C. Syntaxin 4 up-regulation increases efficiency of insulin release in pancreatic islets from humans with and without type 2 diabetes mellitus. J Clin Endocrinol Metab. 2014; 99:E866-870.
27. Stull N D, Breite A, McCarthy R, Tersey S A, Mirmira R G. Mouse islet of Langerhans isolation using a combination of purified collagenase and neutral protease. J Vis Exp. 2012.
28. Reuwer A Q, Nieuwland R, Fernandez I, Goffin V, van Tiel C M, Schaap M C, Berckmans R J, Kastelein J J, Twickler M T. Prolactin does not affect human platelet aggregation or secretion. Thromb Haemost. 2009; 101:1119-1127.
29. Ahn M, Yoder S M, Wang Z, Oh E, Ramalingam L, Tunduguru R, Thurmond D C. The p21-activated kinase (PAK1) is involved in diet-induced beta cell mass expansion and survival in mice and human islets. Diabetologia. 2016; 59:2145-2155.
30. Argués O, Chicote I, Tenbaum S, Puig I, G. Palmer H. Quantitative Procedure to Analyze Nuclear β-Catenin Using Immunofluorescence Tissue Staining. 2014.
31. Eizirik DL, Mandrup-Poulsen T. A choice of death—the signal-transduction of immune-mediated beta-cell apoptosis. Diabetologia. 2001; 44:2115-2133.
32. Corbett J A, Wang J L, Sweetland M A, Lancaster J R, Jr., McDaniel M L. Interleukin 1 beta induces the formation of nitric oxide by beta-cells purified from rodent islets of Langerhans. Evidence for the beta-cell as a source and site of action of nitric oxide. J Clin Invest. 1992; 90:2384-2391.
33. D'Addio F, Maffi P, Vezzulli P, Vergani A, Mello A, Bassi R, Nano R, Falautano M, Coppi E, Finzi G, D'Angelo A, Fermo I, Pellegatta F, La Rosa S, Magnani G, Piemonti L, Falini A, Folli F, Secchi A, Fiorina P. Islet transplantation stabilizes hemostatic abnormalities and cerebral metabolism in individuals with type 1 diabetes. Diabetes Care. 2014; 37:267-276.
34. Figliolini F, Cantaluppi V, De Lena M, Beltramo S, Romagnoli R, Salizzoni M, Melzi R, Nano R, Piemonti L, Tetta C, Biancone L, Camussi G. Isolation, characterization and potential role in beta cell-endothelium cross-talk of extracellular vesicles released from human pancreatic islets. PLoS One. 2014; 9:e102521.
35. Cianciaruso C, Phelps EA, Pasquier M, Hamelin R, Demurtas D, Alibashe Ahmed M, Piemonti L, Hirosue S, Swartz M A, De Palma M, Hubbell J A, Baekkeskov S. Primary Human and Rat beta-Cells Release the Intracellular Autoantigens GAD65, IA-2, and Proinsulin in Exosomes Together With Cytokine-Induced Enhancers of Immunity. Diabetes. 2017; 66:460-473.
36. Klement G L, Yip T T, Cassiola F, Kikuchi L, Cervi D, Podust V, Italiano J E, Wheatley E, Abou-Slaybi A, Bender E, Almog N, Kieran M W, Folkman J. Platelets actively sequester angiogenesis regulators. Blood. 2009; 113:2835-2842.
37. Zhao Y, Jiang Z, Delgado E, Li H, Zhou H, Hu W, Perez-Basterrechea M, Janostakova A, Tan Q, Wang J, Mao M, Yin Z, Zhang Y, Li Y, Li Q, Zhou J, Li Y, Martinez Revuelta E, Maria Garcia-Gala J, Wang H, Perez-Lopez S, Alvarez-Viejo M, Menendez E, Moss T, Guindi E, Otero J. Platelet-Derived Mitochondria Display Embryonic Stem Cell Markers and Improve Pancreatic Islet beta-cell Function in Humans. Stem Cells Transl Med. 2017; 6:1684-1697.
38. Van Belle T L, Taylor P, von Herrath M G. Mouse Models for Type 1 Diabetes. Drug Discov Today Dis Models. 2009; 6:41-45.
39. Wicker L S, Clark J, Fraser H I, Garner V E, Gonzalez-Munoz A, Healy B, Howlett S, Hunter K, Rainbow D, Rosa R L, Smink LJ, Todd J A, Peterson L B. Type 1 diabetes genes and pathways shared by humans and NOD mice. J Autoimmun. 2005; 25 Suppl:29-33.
40. Kabekkodu S P, Bhat S, Radhakrishnan R, Aithal A, Mascarenhas R, Pandey D, Rai L, Kushtagi P, Mundyat G P, Satyamoorthy K. DNA promoter methylation-dependent transcription of the double C2-like domain 13 (DOC2B) gene regulates tumor growth in human cervical cancer. J Biol Chem. 2014; 289:10637-10649.

1. A method of diagnosing type 1 diabetes (T1 D) or pre-T1 D in vivo at an early stage in a subject or assessing the risk of T1 D or pre-T1 D in a subject, comprising:
detecting the level of DOC2B expression in a biological sample collected from the subject, and
comparing the level of DOC2B expression with that of a healthy, control subject or with a pre-set threshold level, wherein a reduced level of DOC2B expression indicates that the subject is suffering from or at an elevated risk of suffering from T1 D or pre-T1 D.

2. A method of treating T1 D or pre-T1 D or delaying the onset of T1 D or pre-T1 D in a subject, comprising:
detecting the level of DOC2B expression in a biological sample collected from the subject,
comparing the level of DOC2B expression with that of a healthy, control subject or with a pre-set threshold level, and
administering one or more T1 D treatments to the subject, if the subject is determined to have a reduced level of DOC2B expression.

3. The method of claim 2, wherein the one or more T1 D treatments include transplanting healthy, functional β cells or pancreatic islets to the subject.

4. A method of assessing early stage pancreatic β-cell destruction or loss of functional β-cells in a subject, comprising:
detecting the level of DOC2B expression in a biological sample collected from the subject, and
comparing the level of DOC2B expression with that of a healthy, control subject or with a pre-set threshold level, wherein a reduced level of DOC2B expression indicates pancreatic β-cell destruction or loss of functional β-cells in the subject.

5. The method of any one of claims 1-4, wherein the biological sample includes blood, plasma, serum, platelets, and pancreatic islets.

6. The method of any one of claims 1-5, wherein detecting the level of DOC2B expression comprises detecting the level of DOC2B protein or the level of DOC2B mRNA in the biological sample.

7. The method of any one of claims 1-6, wherein the level of DOC2B protein is determined by ELISA.

8. The method of claim 7, where the antibody used in ELISA binds to human DOC2B amino acid sequence residues 79-99, 96-116, 249-267 or 23-116.

9. The method of any one of claims 1-8, wherein the level of DOC2B expression is reduced by at least 20%, at least 30%, at least 40%, at least 50%, at least 60%, at least 70%, at least 80%, at least 90%, or at least 95%.

10. The method of any one of claims 1-9, wherein the subject is human.

11. The method of any one of claims 1-10, wherein a reduced level of DOC2B expression is detected prior to onset of T1D or pre-T1D in the subject.


SEQUENCE LISTING (10 sequences)

SEQ ID NO: 1 - protein, 412 aa, Mus musculus
MTLRRRGEKATISIQEHMAIDVCPGPIRPIKQISDYFPRFPRGLPPTAAPRAPAPPDAPARSPAASASPRSPSDGARDDDEDVDQLFGAYGASPGPSPGPSPARPPAKPPEDEPDVDGYESDDCTALGTLDFSLLYDQENNALHCTISKAKGLKPMDHNGLADPYVKLHLLPGASKANKLRTKTLRNTLNPSWNETLTYYGITDEDMVRKTLRISVCDEDKFRHNEFIGETRVPLKKLKPNHTKTFSICLEKQLPVDKAEDKSLEERGRILISLKYSSQKQGLLVGIVRCAHLAAMDANGYSDPYVKTYLKPDVDKKSKHKTAVKKKTLNPEFNEEFCYEIKHGDLAKKTLEVTVWDYDIGKSNDFIGGVVLGINAKGERLKHWFDCLKNKDKRIERWHTLTNELPGAVLSD

SEQ ID NO: 2 - protein, 412 aa, Rattus norvegicus
MTLRRRGEKATISIQEHMAIDVCPGPIRPIKQISDYFPRFPRGLPPTAAPRASAPPDAPARSPAATAGPRSPSDGARDDDEDVDQLFGAYGASPGPSPGPSPVRPPAKPPEDEPDADGYESDDCTALGTLDFSLLYDQENNALHCTISKAKGLKPMDHNGLADPYVKLHLLPGASKANKLRTKTLRNTLNPSWNETLTYYGITDEDMIRKTLRISVCDEDKFRHNEFIGETRVPLKKLKPNHTKTFSICLEKQLPVDKAEDKSLEERGRILISLKYSSQKQGLLVGIVRCAHLAAMDANGYSDPYVKTYLKPDVDKKSKHKTAVKKKTLNPEFNEEFCYEIKHGDLAKKTLEVTVWDYDIGKSNDFIGGVVLGINAKGERLKHWFDCLKNKDKRIERWHTLTNEIPGAVLSD

SEQ ID NO: 3 - protein, 412 aa, Homo sapiens
MTLRRRGEKATISIQEHMAIDVCPGPIRPIKQISDYFPRFPRGLPPDAGPRAAAPPDAPARPAVAGAGRRSPSDGAREDDEDVDQLFGAYGSSPGPSPGPSPARPPAKPPEDEPDADGYESDDCTALGTLDFSLLYDQENNALHCTITKAKGLKPMDHNGLADPYVKLHLLPGASKANKLRTKTLRNTLNPTWNETLTYYGITDEDMIRKTLRISVCDEDKFRHNEFIGETRVPLKKLKPNHTKTFSICLEKQLPVDKTEDKSLEERGRILISLKYSSQKQGLLVGIVRCAHLAAMDANGYSDPYVKTYLRPDVDKKSKHKTAVKKKTLNPEFNEEFCYEIKHGDLAKKSLEVTVWDYDIGKSNDFIGGVVLGIHAKGERLKHWFDCLKNKDKRIERWHTLTSELPGAVLSD

SEQ ID NO: 4 - protein, 405 aa, Mus musculus
MRGRRGDRMTINIQEHMAINVCPGPIRPIRQISDYFPRRGPGPEGGGGGGGTGCGEAPAHLAPLALAPPAALLGATTPDDGAEVDSYDSDDTTALGTLEFDLLYDQASCMLHCRILRAKGLKPMDFNGLADPYVKLHLLPGACKANKLKTKTQRNTLNPVWNEELTYSGITDDDITHKVLRISVCDEDKLSHNEFIGEIRVPLRRLKPSQKKHFNICLERQVPLPSPSSMSAALRGISCYLKELEQAEQGPGLLEERGRILLSLSYSSRRHGLLVGIVRCAHLAAMDVNGYSDPYVKTYLRPDVDKKSKHKTCVKKKTLNPEFNEEFFYEIELSTLATKTLEVTVWDYDIGKSNDFIGGVSLGPGARGEAQKHWNDCLHQPDTALERWHTLTSELPPAAGAYPLA

SEQ ID NO: 5 - protein, 403 aa, Rattus norvegicus
MRGRRGDRMTINIQEHMAINVCPGPIRPIRQISDYFPRRGPGPEGGGGGRGFGEAPAHLAPLALAPPAALLGATTPDDGAEVDSYDSDDTTALGTLEFDLLYDQASCMLHCRILRAKGLKPMDFNGLADPYVKLHLLPGACKANKLKTKTQRNTLNPVWNEELTYSGITDDDITHKVLRISVCDEDKLSHNEFIGEIRVPLRRLKPSQKKHFNICLERQVPLPSPSSMSAALRGISCYLKELEQAEQGPGLLEERGRILLSLSYSSRRHGLLVGIVRCAHLAAMDVNGYSDPYVKTYLRPDVDKKSKHKTCVKKKTLNPEFNEEFFYEMELSTLATKTLEVTVWDYDIGKSNDFIGGVSLGPGARGEAQKHWRDCLHQPDTAVERWHTLTSELPPAAGALPLA

SEQ ID NO: 6 - protein, 400 aa, Homo sapiens
MRGRRGDRMTINIQEHMAINVCPGPIRPIRQISDYFPRGPGPEGGGGGGGEAPAHLVPLALAPPAALLGATTPEDGAEVDSYDSDDATALGTLEFDLLYDRASCTLHCSILRAKGLKPMDFNGLADPYVKLHLLPGACKANKLKTKTQRNTLNPVWNEDLTYSGITDDDITHKVLRIAVCDEDKLSHNEFIGEIRVPLRRLKPSQKKHFNICLERQVPLASPSSMSAALRGISCYLKELEQAEQGQGLLEERGRILLSLSYSSRRRGLLVGILRCAHLAAMDVNGYSDPYVKTYLRPDVDKKSKHKTCVKKKTLNPEFNEEFFYEIELSTLATKTLEVTVWDYDIGKSNDFIGGVSLGPGARGEARKHWSDCLQQPDAALERWHTLTSELPPAAGALSSA

SEQ ID NO: 7 - protein, 379 aa, Artificial Sequence
  Consensus
  MISC_FEATURE (7)..(7): X=E or D
  MISC_FEATURE (20)..(20): X=D or N
  MISC_FEATURE (55)..(55): X=D or E
  MISC_FEATURE (78)..(78): X=D or E
  MISC_FEATURE (82)..(82): X=D or E
  MISC_FEATURE (87)..(87): X=D or E
  MISC_FEATURE (98)..(98): X=D or E
  MISC_FEATURE (172)..(172): X=D or E
  MISC_FEATURE (357)..(357): X=N or Q
MRRRRGXRATINIQEHMAIXVCPGPIRPIRQISDYFPRFGRGLEGGAGGRAAPGXAPARLAPAAAAPRAALDGARTPXDEAXVDGYXSDDCTALGTLXFDLLYDQANNALHCTILRAKGLKPMDHNGLADPYVKLHLLPGACKANKLRTKTQRNTLNPVWNETLTYSGITDXDITRKTLRISVCDEDKLRHNEFIGEIRVPLRRLKPNQKKHFNICLERQLPLDKPEDKSLEERGRILISLKYSSRRQGLLVGIVRCAHLAAMDANGYSDPYVKTYLRPDVDKKSKHKTAVKKKTLNPEFHEEFCYEIEHGDLAKKTLEVTVWDYDIGKSNDFIGGVSLGIGARGEALKHWFDCLKXKDKAIERWHTLTSELPGAAGAD

SEQ ID NO: 8 - protein, 408 aa, Artificial Sequence
  Consensus
MTLRRRGEKATISIQEHMAIDVCPGPIRPIKQISDYFPRFPRGLPPTAAPRAAPPDAPARSPAAAGPRSPSDGARDDEDVDQLFGAYGASPGPSPGPSPARPPAKPPEDEPDADGYESDDCTALGTLDFSLLYDQENNALHCTISKAKGLKPMDHNGLADPYVKLHLLPGASKANKLRTKTLRNTLNPSWNETLTYYGITDEDMRKTLRISVCDEDKFRHNEFIGETRVPLKKLKPNHTKTFSICLEKQLPVDKAEDKSLEERGRILISLKYSSQKQGLLVGIVRCAHLAAMDANGYSDPYVKTYLKPDVDKKSKHKTAVKKKTLNPEFNEEFCYEIKHGDLAKKTLEVTVWDYDIGKSNDFIGGVVLGINAKGERLKHWFDCLKNKDKRIERWHTLTNELPGAVLSD

SEQ ID NO: 9 - DNA, 20 bp, artificial
  Synthetic
ccagtaaggcaaataagctc

SEQ ID NO: 10 - DNA, 17 bp, artificial
  Synthetic
gggtttcagcttcttca